(19) United States
(12) Patent Application Publication
(10) Pub. No.: US 2008/0009913 A1
(43) Pub. Date: Jan. 10, 2008

(54) METHODS AND APPARATUS FOR THE TREATMENT OF EATING DISORDERS USING ELECTRICAL IMPULSE INTERVENTION

(75) Inventors: Joseph P. ERRICO, Green Brook, NJ (US); Steven MENDEZ, Chester, NJ (US)

Correspondence Address: KAPLAN GILMAN GIBSON & DERNIER L.L.P., 900 ROUTE 9 NORTH, WOODBRIDGE, NJ, 07095, US

(73) Assignee: ELECTROCORE, INC., Summit, NJ (US)

(21) Appl. No.: 11/754,522

(22) Filed: May 29, 2007

Related U.S. Application Data
(60) Provisional application No. 60/818,909, filed on Jul. 6, 2006.

(51) Int. Cl. A61N 1/36

(52) U.S. Cl. 607/40

(57) ABSTRACT

Devices and methods for treating patients suffering from an eating disorder, such as obesity and/or pathologies resulting in obesity, by regulating sensations affecting food consumption. The devices and methods may facilitate appropriate caloric intake, thereby inducing weight loss, by simulating, stimulating, amplifying, blocking and/or modulating signals in the gastrointestinal (GI) tract and/or nerves innervating the GI tract, to manage sensations of hunger and satiety, such as controlling hunger by signaling the gastrointestinal tract and/or gastrointestinal nerves when different hunger sensations are detected.

### CROSS REFERENCE TO RELATED APPLICATIONS

This application is based on and claims the benefit of U.S. Provisional Patent Application No. 60/818,909, filed Jul. 6, 2006, the entire disclosure of which is hereby incorporated by reference.

### BACKGROUND OF THE INVENTION

The present invention relates to the field of delivery of electrical impulses to bodily tissues for therapeutic purposes, and more specifically to devices and methods for treating patients suffering from one or more eating disorders, such as obesity and/or pathologies resulting in obesity.

The use of electrical stimulation for treatment of medical conditions has been well known in the art for nearly two thousand years. Roman physicians are reported to have used electric eels for treating headaches and pain associated with gout. In 1760, John Wesley applied the primitive rudimentary electrical device, the Leyden Jar, to therapeutic purposes hoping to shock patients suffering from paralysis, convulsions, seizures, headaches, angina, and sciatica.

It was not until Luigi Galvani, in 1791, that a disciplined study of the effects of electricity on muscles and nerves was done in a scientifically rigorous manner. In 1793, Alessandro Volta furthered this work when he reported that muscle contraction could be forced to occur when an electrified metal was placed in the vicinity of a motor nerve and the muscle innervated by that nerve.

One of the most successful modern applications of this basic understanding of the relationship between muscle and nerves is the cardiac pacemaker. Although its roots extend back into the 1800's, it wasn't until 1950 that the first practical, albeit external and bulky pacemaker was developed. Dr. Rune Elqvist developed the first truly functional, wearable pacemaker in 1957. Shortly thereafter, in 1960, the first fully implanted pacemaker was developed.

Around this time, it was also found that the electrical leads could be connected to the heart through veins, which eliminated the need to open the chest cavity and attach the lead to the heart wall. In 1975 the introduction of the lithium-iodide battery prolonged the battery life of a pacemaker from a few months to more than a decade. The modern pacemaker can treat a variety of different signaling pathologies in the cardiac muscle, and can serve as a defibrillator as well (see U.S. Pat. No. 6,738,667 to Deno, et al., the disclosure of which is incorporated herein by reference).

Another application of electrical stimulation of nerves has been the treatment of radiating pain in the lower extremities by means of stimulation of the sacral nerve roots at the bottom of the spinal cord (see U.S. Pat. No. 6,871,099 to Whitehurst, et al., the disclosure of which is incorporated herein by reference).

A further application is disclosed in U.S. Pat. No. 6,957,106 (“'106”)to Schuler, et al., entitled, “Implantable method to regulate blood pressure by means of coded nerve signals,” which is incorporated in its entirety by reference. The '106 patent states that, “the electrical action for regulating cardiovascular blood pressure emerges from the medullopontine area via the vagus nerve bundle.” Affecting the electrical action of the vagus nerve bundle therefore may affect regulation of blood pressure, making the vagus nerve a further subject of electrical stimulation study.

Most of the life support control of the human or animal body is via the vagus (or tenth cranial) nerve that exits from the medulla oblongata. Paralysis or severing the two vagus nerves at the level of the medulla or neck is rapidly fatal. This nerve is actually a long bundle of afferent and efferent neurons that travels over the internal body to most organs, including the stomach. The vagus nerve emerges from each side of the medulla and travels different routes to the same target organs. For instance, the left vagus innervates the antero-superior surface of the stomach.

The nerves innervating the stomach are the terminal branches of the right and left vagi, the former being distributed upon the back, and the latter upon the front part of the organ. A great number of branches from the celiac plexus of the sympathetic are also distributed to it. Nerve plexuses are found in the submucous coat and between the layers of the muscular coat as in the intestine. From these plexuses, fibrils are distributed to the muscular tissue and the mucous membrane.

The stomach is the most dilated part of the digestive tube, and is situated between the end of the esophagus and the beginning of the small intestine. The stomach presents two openings, two borders or curvatures, and two surfaces. When the stomach is in the contracted condition, its surfaces are directed upward and downward respectively, but when the viscus is distended they are directed forward, and backward. They may therefore be described as antero-superior and postero-inferior.

Of the antero-superior surface, the left half is in contact with the diaphragm, which separates it from the base of the left lung, the pericardium, and the seventh, eighth, and ninth ribs, and intercostal spaces of the left side. The right half is in relation with the left and quadrate lobes of the liver and with the anterior abdominal wall. When the stomach is empty, the transverse colon may lie on the front part of this surface. The whole surface is covered by the peritoneum.

The postero-inferior surface is in relation with the diaphragm, the spleen, the left suprarenal gland, the upper part of the front of the left kidney, the anterior surface of the pancreas, the left colic flexure, and the upper layer of the transverse mesocolon. These structures form a shallow bed, the stomach bed, on which the viscus rests. The transverse mesocolon separates the stomach from the duodenojejunal flexure and small intestine. The postero-inferior surface is covered by the peritoneum, except over a small area close to the cardiac orifice; this area is limited by the lines of attachment of the gastrophrenic ligament, and lies in apposition with the diaphragm, and frequently with the upper portion of the left suprarenal gland.

With respect to the component parts of the stomach, which are illustrated in FIG. 3A, a plane passing through the incisura angularis on the lesser curvature and the left limit of the opposed dilatation on the greater curvature divides the stomach into a left portion or body and a right or pyloric portion. The left portion of the body is known as the fundus, and is marked off from the remainder of the body by a plane passing horizontally through the cardiac orifice. The pyloric portion is divided by a plane through the sulcus intermedius at right angles to the long axis of this portion; the part to the right of this plane is the pyloric antrum.

Physiologically, the stomach acts as a gateway to food consumption, and hence weight gain, leading to overweight conditions and obesity. Many people have an insatiable desire to eat and consequently overeat, leading to overweight conditions and sometimes obesity. An individual is considered overweight if the person has a score of 25 or more on the body mass index (BMI), a measurement tool used to determine excess body weight. A person's BMI score is the ratio of his weight in kilograms to the square of his height in meters (i.e., kg/m²). Persons having a BMI score of 30 or more qualify as obese, whereas those with BMI scores of 40 and over are considered severely obese.

As of 2002, overweight conditions and obesity were estimated to affect over 127 million adults and over 9 million children in the United States alone, and several hundreds of millions of people worldwide. Of the approximately 127 million overweight adults in the U.S., around 60 million are considered obese, and 9 million of these 60 million qualify as severely obese. Percentagewise, that means that 64.5% of U.S. adults are overweight, 30.5% are obese, and 4.7% are severely obese.

The Centers for Disease Control (CDC) refers to obesity and overweight conditions as chronic conditions that have turned into an epidemic. Being overweight, and to a greater extent obese, increases the risk of many health conditions and diseases including hypertension, dyslipidemia, type-2 diabetes, coronary heart disease, stroke, gallbladder disease, osteoarthritis, sleep apnea, respiratory problems, and even some cancers (endometrial, breast and colon). Although there are many efforts to reduce the prevalence of overweight conditions and obesity, data indicate that the number of adults and children becoming overweight and obese is growing.

Overweight conditions and obesity also increase government and medical expenditures. In 2003 the CDC concluded that taxpayers paid $39 billion in obesity-related medical costs, covering more than half of the $75 billion in obesity-related medical costs that year. This amount is for treating obesity-related medical problems through Medicare and Medicaid. Obesity-related expenditures account for about 10% of the total medical expenditures in the U.S. The State of California, alone, spends almost $7.7 billion on obesity-related medical treatment each year.

When it comes to obesity, the saying holds true that we are what we eat. Food consumption provides a body with energy, measured in, and referred to as, calories, that is needed for the body to function. The body's metabolism converts calories into fuel for physical activity. Depending on the level of physical activity relative to caloric consumption, calories will either be metabolized as fuel for immediate use, or stored as fat for future use. When the body runs low on fuel for immediate use, a release of appetite hormones may cause the individual to experience hunger and therefore to eat. Eating in turn releases hormones that trigger satiety, which should cause the individual to stop eating.

Satiety, or the feeling of fullness and disappearance of appetite after a meal, is a process mediated by the ventromedial nucleus in the hypothalamus, known as the “satiety center.” Various hormones, first of all cholecystokinin, have been implicated in conveying the feeling of satiety to the brain. Leptin increases on satiety, while ghrelin increases when the stomach is empty. Therefore, satiety refers to the psychological feeling of satisfaction after eating rather than to the physical feeling of being engorged, i.e., the feeling of physical fullness after eating a very large meal. Satiety directly influences feelings of appetite that are generated in the limbic system, and hunger that is controlled by neurohormones, especially serotonin in the lateral hypothalamus. Preferably, satiety causes an individual to stop eating.

Leptin, in conjunction with other hormones, is used by the body to regulate appetite and metabolism. More specifically, leptin is a 16 kDa protein hormone that plays a key role in regulating energy intake and energy expenditure. Leptin is produced by the expression of the Ob(Lep) gene, located on chromosome 7 in humans, by adipose tissue (i.e., it is released from fat cells). Adipose tissue is loose connective tissue composed of adipocytes, the main role of which is to store energy in the form of fat, although it also cushions and insulates the body and performs an important endocrine function in producing hormones such as leptin, resistin and TNFα.

Leptin interacts with six types of receptors (LepRa-LepRf). LepRb is the only receptor isoform that contains active intracellular signaling domains and is present in a number of hypothalamic nuclei, where it exerts its effects. Importantly, leptin binds to the ventral medial nucleus of the hypothalamus, or “satiety center” as mentioned above. The binding of leptin to this nucleus signals to the brain that the body has had enough to eat—a sensation of satiety. A very small group of humans, mostly arising from inbred populations, are mutant for the leptin gene. These people eat nearly constantly, and may be more than 100 pounds (45 kg) overweight by the age of 7.

Leptin works by inhibiting the activity of neurons that contain neuropeptide Y (NPY) and agouti-related peptide (AgRP), and by increasing the activity of neurons expressing α-melanocyte-stimulating hormone (α-MSH) . The NPY neurons are a key element in the regulation of appetite; small doses of NPY injected into the brains of experimental animals stimulates feeding, while selective destruction of the NPY neurons in mice causes them to become anorexic. Conversely, α-MSH is an important mediator of satiety, and differences in the gene for the receptor at which α-MSH acts in the brain are linked to obesity in humans. Leptin is also regulated (downward) by melatonin during the night.

Once leptin has bound to the Ob-Rb receptor, it activates the molecule stat3, which is phosphorylated and travels to the ventral medial nucleus, it is presumed, to effect changes in gene expression. One of the main effects on gene expression is the down-regulation of the expression of endocannabinoids, which are responsible for increasing appetite, among their many other functions. There are other intracellular pathways activated by leptin, but less is known about how they function in this system. In response to leptin, receptor neurons have been shown to remodel themselves, changing the number and types of synapses that fire onto them.

Leptin is released by fat cells in amounts mirroring overall body fat stores. Thus, circulating leptin levels give the brain a reading of energy storage for the purposes of regulating appetite and metabolism. Although leptin is a circulating signal that reduces appetite, in general, the amount of leptin produced increases with weight gain, so obese people have an unusually high circulating concentration of leptin. The increase in leptin levels should result in increased signals for the body to intake less food. However, overweight and obese people seem to be resistant to the signals sent by leptin, contributing to their excessive food consumption.

Some obese people are said to be resistant to the effects of leptin in much the same way that people with type-2 diabetes are resistant to the effects of insulin. In general, obesity develops when people take in more energy than they use over a prolonged period of time. In leptin-resistant obese people, this excess food intake is not driven by hunger signals and occurs in spite of the anti-appetite signals from circulating leptin. The high sustained concentrations of leptin from the enlarged fat stores result in the cells that respond to leptin becoming desensitized.

Excessive caloric intake creates an excess energy imbalance wherein there is a consumption of calories without a proportional use of calories, such as by physical activity. Recurring excess energy imbalances over a long period of time are what ultimately cause overweight conditions and obesity. There are many factors that affect the dynamics of this energy imbalance for a given individual, including the individual's genetics, environment, eating choices, physical activity choices, diseases and drug use. Tragically, many overweight and obese people, although aware of their problem, believe that it is beyond their control.

There have been numerous attempts to curb appetites and increase physical activity in an effort to control weight gain and stimulate weight loss. These attempts include drugs for appetite suppression, diet plans, risky surgeries, and hypnosis. Though many of these weight control methods have shown initial results, once weight loss begins to plateau, an individual often reverts back to previous behavior which causes weight gain.

A number of electrical devices and processes are taught in the art for attempting to control an individual's food intake and/or various aspects of the digestive process in an effort to treat eating or digestive disorders. Some prior art references focus on the movement of food. Chen, et al., U.S. Pat. No. 5,690,691, discloses a gastric pacemaker implantable in the gastrointestinal tract to deliver a phased electrical stimulation to pace peristalsis to enhance or accelerate peristaltic movement through the gastric tract or to attenuate the peristaltic movement to treat such conditions eating disorders or diarrhea. Likewise, Terry, Jr., et al., U.S. Pat. No. 5,540,730, discloses an apparatus and method of treating motility disorders by selectively stimulating a patient's vagus nerve to modulate electrical activity of the nerve and to thereby cause a selective release or suppression of excitatory or inhibitory transmitters. One embodiment employs the manual or automatic activation of an implanted device for selective modulation. Similarly, Cigaina, U.S. Pat. No. 5,423,872, discloses a process and device for treating obesity and syndromes related to motor disorders of the stomach by altering the natural gastric motility of a patient by electrical stimulation to prevent emptying or to slow down food transit.

U.S. Patent Application Number 20050222637, to Chen, entitled Tachygastrial Electrical Stimulation, which is incorporated by reference herein, discloses treating obesity by “artificially altering, by means of electrical pulses for preset periods of time, the natural gastric motility of the patient to prevent the emptying of or to slow down gastric transit through the stomach to increase the feeling of satiety and/or to accelerate intestinal transit to reduce absorption time within the intestinal tract. More specifically, the electrical stimulation induces tachygastria, which inhibits gastric motility, yields gastric distention, and delays gastric emptying. The tachygastrial electrical stimulation of the stomach, or other portions of the gastrointestinal tract, includes relatively long pulse widths, with lengths of up to 500 milliseconds.”

Other prior art references focus on sensory aspects of food consumption. Zikria, U.S. Pat. No. 6,564,101, discloses a system for controlling a patient's appetite using an electrical signal controller that sends electrical signals to the fundus of the patient's stomach, wherein the controller generates substantially continuous low voltage stimulation with varying periodicity as determined by the individual's specific physiology, anatomy and/or psychology.

Wernicke, et al., U.S. Pat. No. 5,188,104 (“'104”), which is incorporated by reference herein, discloses a method and apparatus of using electrical stimulation of the vagus nerve to treat patients with compulsive eating disorders. The '104 patent proposes “detecting a preselected event indicative of an imminent need for treatment of the specific eating disorder of interest, and responding to the detected occurrence of the preselected event by applying a predetermined stimulating signal to the patient's vagus nerve appropriate to alleviate the effect of the eating disorder of interest.”

The '104 patent indicates that in cases of compulsive excessive eating, “the stimulating signal is predetermined to produce a sensation of satiety in the patient,” whereas, if “the disorder is compulsive refusal to eat (anorexia nervosa), the stimulating signal is predetermined to produce a sensation of hunger or to suppress satiety in the patient.”

In the '104 patent, the preselected event may be, for example, “a specified level of food consumption by the patient within a set interval of time, or the commencement of a customary mealtime according to the patient's circadian cycle, or the passage of each of a sequence of preset intervals of time, or the patient's own recognition of the need for treatment by voluntarily initiating the application of the stimulating signal to the vagus nerve.” The '104 patent suggests detecting the occurrence of the preselected event “by summing the number of swallows of food by the patient within the set interval of time.”

However, none of the aforementioned devices is sufficient for effective treatment of obesity-related eating disorders. Accordingly, there are needs in the art for new products and methods for treating the mediators of obesity that contribute to excessive food consumption.

### SUMMARY OF THE INVENTION

The present invention involves products and methods for regulating sensations affecting food consumption, as a treatment for patients suffering from one or more eating disorders, such as obesity and/or pathologies resulting in obesity, utilizing an electrical signal that may be applied to the gastrointestinal tract and/or GI tract nerves to temporarily stimulate, amplify, block and/or modulate the nerve signals associated with sensations of satiety and/or hunger. The present invention encompasses treatment of pathologies resulting in obesity, both general and severe obesity, such as in patients with thyroid pathologies and those suffering from side effects of medications or Cushing's disease. This treatment of obesity may accompany treatment for other conditions, such as depression, that also may occur in situations of weight gain.

In a first embodiment, the present invention contemplates a method of regulating sensations affecting food consumption and/or treating eating disorders, primarily obesity and/or pathologies resulting in obesity, using an electrical signal detection and delivery device (ESDD) that detects patient-generated signals associated with food consumption, models the patient-generated signals, and delivers one or more electrical impulses to at least one selected region of the GI tract and/or nerves innervating the GI tract, to stimulate, amplify, block and/or modulate signals associated with sensations of satiety and/or hunger. The method also may include programming the ESDD device to perform specific sensing and signaling functions.

In a second embodiment, the present invention contemplates an electrical signal detection and delivery device for regulating sensations affecting food consumption such as sensations of satiety and/or hunger. The device may include a sensor that may detect patient-generated signals (PGS) associated with food consumption; a control unit that may model, stimulate, amplify and/or block the patient-generated signals; an electrical impulse generator that delivers one or more electrical impulses to at least one selected region of the GI tract and/or nerves innervating the GI tract; electrodes and/or leads for sensing PGS and/or delivering electrical impulses to stimulate, amplify, block and/or modulate PGS associated with food consumption; and a power supply. The ESDD device also may include a receiver, or optionally a transceiver, for communication of information, settings, data, etc., between a programming unit and the control unit.

In distinct preferred embodiments, the impulses are applied in a manner that blocks patient-generated hunger sensation signals and/or simulates or amplifies patient-generated satiety sensation signals. In this regard, the simulation of patient-generated satiety sensation signals involves substantially copying the patient's own signals associated with particular sensations and feeding back those signals to the patient when appropriate or desirable. Such simulation may involve amplifying existing signals or providing signals where none exist at the time they are needed or desired. It shall be understood that the activation of such impulses may be directed, depending on the embodiment, automatically or manually by a patient suffering from obesity or the patient's healthcare attendant, such as a doctor, nurse, or primary care giver.

Whereas the present invention is concerned primarily with treating obesity by inducing weight loss through reduced food consumption, the present invention also applies to severe cases of anorexia, where weight gain through increased food consumption is desired. In cases where weight gain is desired, the impulses may be applied in a manner that simulates or amplifies patient-generated hunger sensation signals and/or blocks patient-generated satiety sensation signals.

The patient-generated signals may be detected, and the impulses may be applied, by positioning leads on the GI tract and/or nerves innervating the GI tract, such as in the fundus area of the stomach, that transmit sensations of hunger and satiety, such as the terminal branches of the left and right vagi, and the branches from the celiac plexus of the sympathetic. Leads may be positioned proximally or distally to include, respectively, more or less tissue affected by the signal. It shall also be understood that leadless impulses as shown in the art may also be utilized for applying impulses to the target regions.

The mechanisms by which the appropriate impulse is applied to the selected region of the GI tract and/or GI tract nerves can include positioning the distal ends of an electrical lead or leads in the vicinity of the nervous tissue controlling sensations of hunger and satiety, where the leads are coupled to an implantable or external electrical impulse generating device. The electric field generated at the distal tip of the lead creates a field of effect that permeates the target nerve fibers and causes the stimulating, blocking and/or modulating of signals to the subject tissue.

The application of electrical impulses, either to the GI tract or GI tract nerves to stimulate, block and/or modulate the sensations of hunger or satiety is more completely described in the following detailed description of the invention, with reference to the drawings provided herewith, and in claims appended hereto.

Other aspects, features, advantages, etc. will become apparent to one skilled in the art when the description of the invention herein is taken in conjunction with the accompanying drawings.

### BRIEF DESCRIPTION OF THE DRAWINGS

For the purposes of illustrating the various aspects of the invention, there are shown in the drawings forms that are presently preferred, it being understood, however, that the invention is not limited by or to the precise data, methodologies, arrangements and instrumentalities shown.

FIG. 1 is a diagrammatic view of the sympathetic and parasympathetic nerve systems.

FIG. 2 is a cross-sectional anatomical illustration of selected portions of a neck, thoracic and abdominal region.

FIG. 3A illustrates a simplified view of a stomach and its parts.

FIG. 3B illustrates a simplified view of a stomach with an exemplary electrical signal detection and delivery device attached proximate the vagus nerve shown in FIGS. 1 and 2.

FIG. 4 illustrates an exemplary electrical voltage/current profile for a stimulating, blocking and/or modulating impulse applied to a portion or portions of the GI tract and/or nerves innervating the GI tract, in accordance with an embodiment of the present invention.

FIGS. 5A and 5B illustrate an exemplary complex copper micro-coil, and a close-up thereof, respectively, for use in accordance with the present invention.

FIG. 6 illustrates a flow diagram of an exemplary implementation of an embodiment of the present invention.

### DETAILED DESCRIPTION OF THE PREFERRED EMBODIMENTS

For the purposes of illustration, forms are shown in the drawings that are preferred, it being understood that the invention is not limited to precise arrangements or instrumentalities shown.

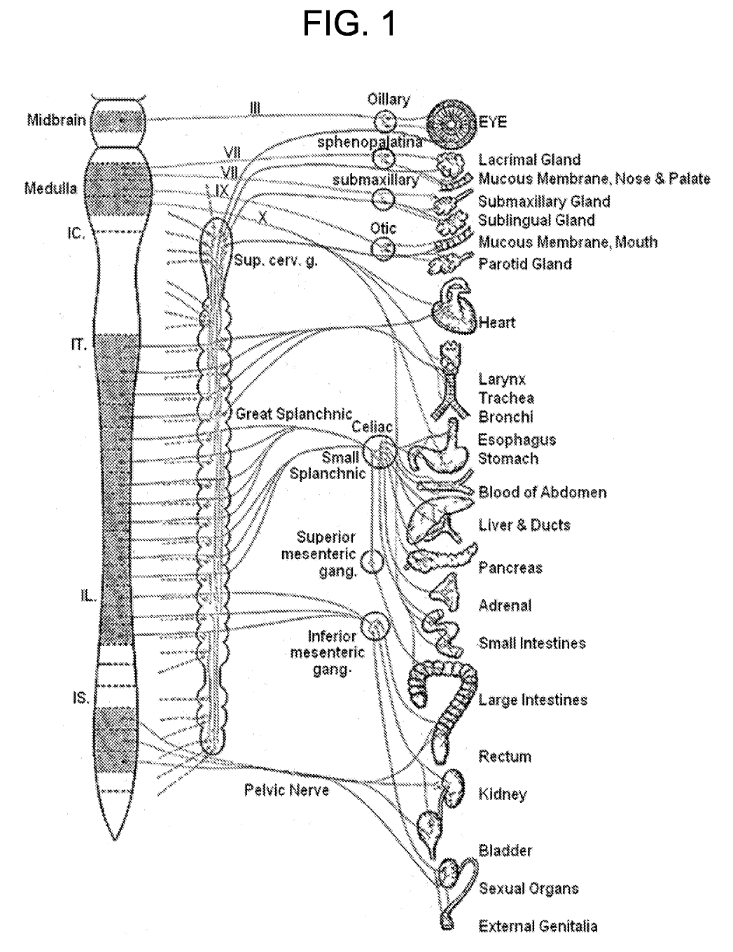

Referring to FIG. 1, a diagrammatic view of the sympathetic and parasympathetic nerve systems is shown. Interestingly, it has been observed in the literature that the nervous system maintains a balance of the signals carried by the sympathetic and parasympathetic nerves. From the sympathetic nerves, the stomach is innervated by the celiac plexus (shown coming from the left). From the parasympathetic nerves (III, VII, VIII, IX, X and Pelvic shown here), the vagus nerve (i.e., X) is shown extending down to the stomach, in addition to the heart, larynx, trachea, bronchi, esophagus, blood of the abdomen, liver & ducts, pancreas, small intestines, and large intestines.

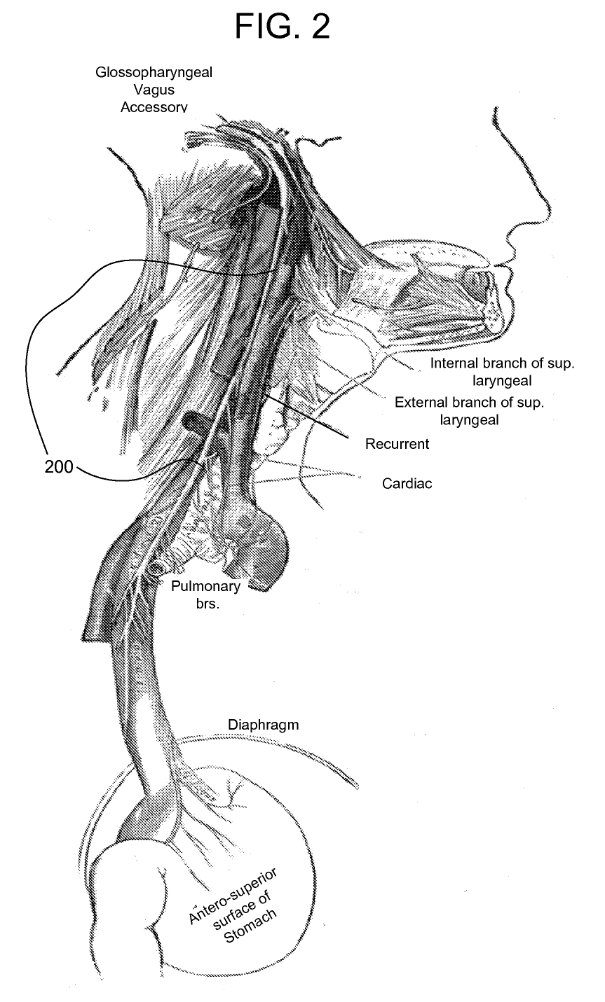

Referring to FIG. 2, a cross-sectional anatomical illustration of selected portions of a neck, thoracic and abdominal region depicts the vagus nerve in more detail. The vagus nerve is composed of motor and sensory fibers. The vagus nerve leaves the cranium and is contained in the same sheath of dura matter with the accessory nerve. The vagus nerve passes down the neck within the carotid sheath to the root of the neck. Parasympathetic innervation of the stomach is mediated by the vagus nerve. The branches of distribution of the vagus nerve include, among others, the superior cardiac, the inferior cardiac, the anterior bronchial and the posterior bronchial branches.

On the right side, the vagus nerve descends by the trachea to the back of the root of the lung, where it spreads out in the inferior cardiac branch and the posterior pulmonary plexus. The right vagus innervates the Sinoatrial node. Parasympathetic hyperstimulation predisposes those affected to bradyarrhythmias. On the left side, the vagus nerve enters the thorax, crosses the left side of the arch of the aorta, forming the superior cardiac branch, and descends behind the root of the left lung, forming the posterior pulmonary plexus. The left vagus when hyperstimulated predisposes the heart to Atrioventricular (AV) blocks.

In mammals, two vagal components have evolved in the brainstem to regulate peripheral parasympathetic functions. The dorsal vagal complex (DVC), consisting of the dorsal motor nucleus (DMNX) and its connections, controls parasympathetic function below the level of the diaphragm, while the ventral vagal complex (VVC), comprised of nucleus ambiguus and nucleus retrofacial, controls functions above the diaphragm in organs such as the heart, thymus and lungs, as well as other glands and tissues of the neck and upper chest, and specialized muscles such as those of the esophageal complex.

The parasympathetic portion of the vagus innervates ganglionic neurons which are located in or adjacent to each target organ. The VVC appears only in mammals and is associated with positive as well as negative regulation of heart rate, bronchial constriction, vocalization and contraction of the facial muscles in relation to emotional states. Generally speaking, this portion of the vagus nerve regulates parasympathetic tone. Muscle tone (residual muscle tension) is the continuous and passive partial contraction of the muscles. The VVC inhibition is released (turned off) in states of alertness.

The parasympathetic tone is balanced in part by sympathetic innervation, which generally speaking supplies signals that, for instance in the case of heart and lungs, tend to expand the myocardium and to relax the bronchial muscles, so that over-contraction and over-constriction, respectively, do not occur. Stimulation of the vagus nerve (up-regulation of tone), such as may occur, for example in shock, results, for instance in the case of heart and lungs, in a heart rate decrease and airway constriction.

In this context, up-regulation is the process by which the specific effect is increased, whereas down-regulation involves a decrease of the effect. On a cellular level, up-regulation is the process by which a cell increases the number of receptors to a given hormone or neurotransmitter to improve its sensitivity to this molecule. A decrease of receptors is called down-regulation.

In accordance with at least one aspect of the present invention, the delivery, in a patient suffering from obesity or being overweight, of an electrical impulse sufficient to simulate, stimulate, amplify, block and/or modulate transmission of signals in the GI tract and/or nerves innervating the GI tract, such as the vagus nerve, will result in regulating sensations associated with satiety and/or hunger. More particularly, such electrical impulse(s) are operable to stimulate, amplify, block and/or modulate transmission of signals to and from the tissues and/or nerves innervating the GI tract, to affect: sensations of hunger, sensations of satiety, sensations of stomach fullness, sensations of stomach emptiness, and sensations of stomach pain. The simulation of patient-generated sensation signals involves substantially copying the patient's own signals associated with particular sensations of the GI tract and feeding back those signals to the patient when appropriate or desirable. Such simulation may involve amplifying existing signals or providing signals where none exist at the time they are needed or desired.

The methods described herein of applying an electrical impulse to a selected region of the GI tract and/or nerves innervating the GI tract may further be refined such that the at least one region may comprise at least one nerve fiber emanating from the patient's tenth cranial nerve (the vagus nerve), and in particular, at least one of the antero-superior and/or postero-inferior surface branches thereof. Likewise, the at least one region may comprise at least one nerve fiber emanating from the patient's sympathetic nerve, and in particular, the celiac plexus.

As necessary, the impulse may be directed to a region of the GI tract and/or GI tract nerves, such as the fundus region of the stomach and/or the vagus nerve, to simulate, stimulate, amplify, block and/or modulate signals in the GI tract branches. As recognized by those having skill in the art, this embodiment should be carefully evaluated prior to use in patients known to have preexisting electrophysiological issues.

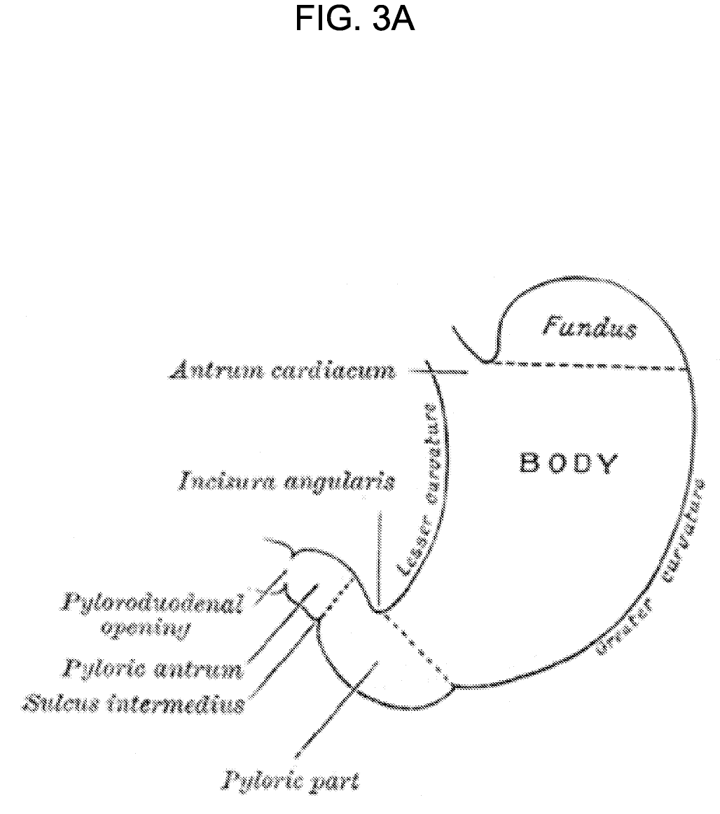

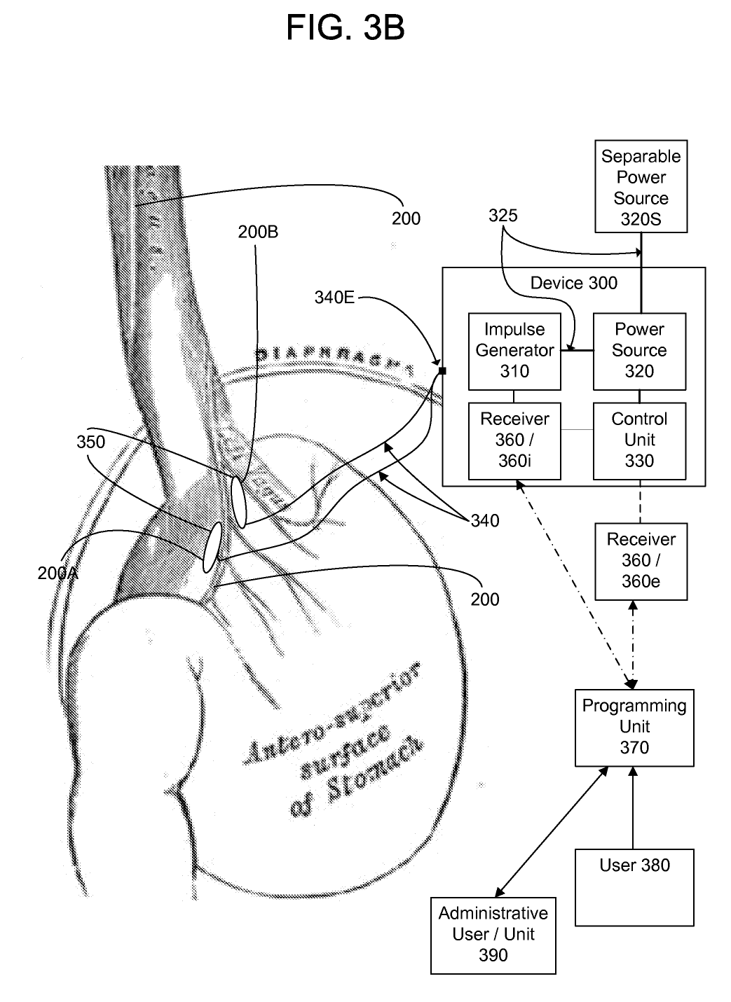

Referring to FIGS. 3A and 3B, FIG. 3A illustrates a simplified view of a stomach and its parts, whereas FIG. 3B illustrates a stomach with an exemplary electrical signal detection and delivery device 300 attached proximate the vagus nerve 200 shown in FIGS. 1 and 2. The electrical signal detection and delivery (ESDD) device 300 detects patient-generated signals (PGS) in the GI tract tissue and/or GI tract nerves. These patient-generated signals are associated with one or more sensations identified by the patient relating to the patient's GI tract activity, such as sensations of hunger, sensations of satiety, sensations of stomach fullness, sensations of stomach emptiness, and sensations of stomach pain. Detected signal patterns may be stored and associated with their physiological sensations (e.g., hunger or satiety). PGS may be monitored and regulated periodically. To induce weight loss through reduced food consumption, ESDD device 300 may block PGS for hunger and simulate (e.g., through stimulation and/or amplification) PGS for satiety.

ESDD device 300 may include an electrical impulse generator 310; a power source 320 coupled to the electrical impulse generator 310; a control unit 330 in communication with the electrical impulse generator 310 and coupled to the power source 320; and electrodes 350 coupled to the electrical impulse generator 310, power source 320, and/or control unit 330, for attachment via leads 340 to one or more selected regions 200A, 200B of the GI tract and/or GI nerves, such as vagus nerve 200 of a mammal.

Power source 320 may couple to the electrical impulse generator 310 and control unit 330 via a power connection 325. While the ESDD 300 requires power to function, the power source 320 may include a removable battery or other separable power source 320S that may not accompany the ESDD 300 at the time of manufacture or sale. Before use of the ESDD 300, the separable power source 320S may be coupled to the power connection 325. Therefore, the present invention also covers an ESDD 300 having a power connection 325 without a power source 320.

Depending on the configuration, each of one electrodes 350 and leads 340 may function to detect patient-generated signals, generate regulating impulses, or both. If a lead 340 is used, it may be preferable to shield the electrode 350, so that electrode 350 functions as a lead wire coupling the lead 340 and ESDD 300. In the context of detection, electrodes 350 and leads 340 may be sensor electrodes and inductive pickup coils. Combined with the control unit 330, sensor electrodes and/or inductive pickup coils may function as examples of sensing means. In the context of regulation, electrodes 350 and leads 340 may be impulse electrodes and inductive impulse coils. Combined with the electrical impulse generator 310 and the control unit 330, impulse electrodes and/or inductive impulse coils may function as examples of signaling means. Coils may be preferable if the desired attachment area is too delicate for attachment of an electrode.

To the extent that a single electrode 350 and/or lead 340 is used to detect signals and generate impulses, the control unit 330 switches the function of the electrode 350 and/or lead 340 when necessary to alternate between sensing and signaling. Switched to the sensing function, the control unit 330 receives input from the electrodes 350 and/or leads 340. Switched to the signaling function, the control unit 330 regulates the signal output of the electrodes 350 and/or leads 340.

The device 300 may be self-contained, as shown, or comprised of various separate, interconnected units. The control unit 330 may control the electrical impulse generator 310 for generation of a signal suitable for stimulating, amplifying, modulating and/or blocking PGS when the signal is applied via the electrodes 350 and/or leads 340 to the GI tract and/or GI tract nerves, such as vagus nerve 200. Via the connections to electrodes 350 and leads 340, the control unit 330 receives and collects sensor information.

The control unit 330 also may have a receiver 360, by which information from a programming unit 370 operable by a user 380 may be received. The receiver 360 may comprise an external driver (360e), or alternatively, an internal driver (360i) whereby control unit 330 may comprise a complete, self-contained implantable unit. Receiver 360 may comprise a transceiver able to transmit information back to the programming unit 370. The programming unit 370 may be outside the body and operable to communicate settings, information and data to and from the control unit 330.

In accordance with a preferred embodiment, ESDD devices 300 in accordance with the present invention are provided in the form of a percutaneous or subcutaneous implant that can be reused by an individual.

For percutaneous use, the ESDD device 300 may be available to the user 380 (e.g., patient or healthcare attendant) as an external appliance, whereby leads 340 and electrodes 350 may be implanted in the patient, but have connection ends 340E traversing the skin for coupling to ESDD device 300. For subcutaneous use, the ESDD device 300 may be surgically implanted, such as in a subcutaneous pocket of the abdomen. Depending on configuration, the ESDD device 300 may be powered and/or recharged from outside the body or may have its own power source 320. By way of example, the ESDD device 300 may be purchased commercially. The ESDD device 300 is preferably programmed with a physician programmer, such as a Model 7432 also available from Medtronic, Inc.

In obese patients, one or more ESDD devices 300 may be implanted in one or more selected regions 200A, 200B of the GI tract area. U.S. Patent Application Publications 2005/0075701 and 2005/0075702, both to Shafer, both of which are incorporated herein by reference, relating to stimulation of neurons of the sympathetic nervous system to attenuate an immune response, contain descriptions of impulse generators that may be applicable to the present invention.

Implantation of the device may be done using known techniques, such as described in U.S. Pat. No. 7,020,531, to Colliou, et al., which is incorporated by reference herein. Colliou, et al. teach attachment of a functional device to a stomach wall, such as a device providing electrical stimulation of the stomach wall. Where necessary, similar or different techniques may be used to attach the device elsewhere besides the stomach.

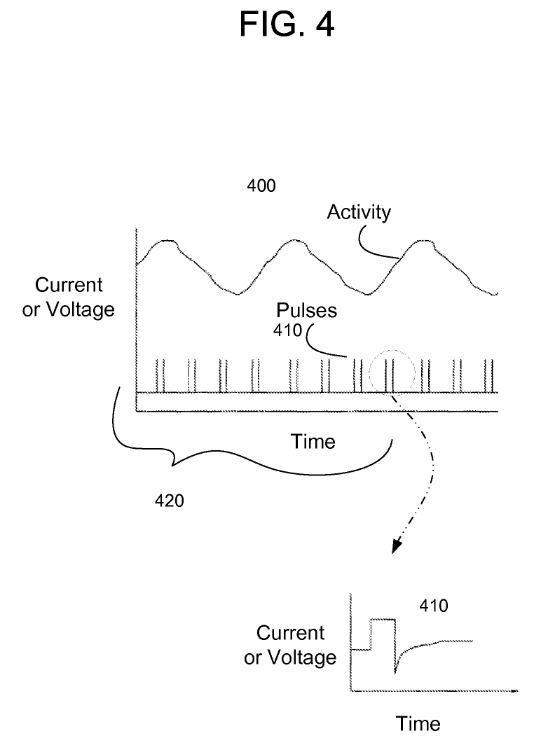

Referring to FIG. 4, an exemplary electrical voltage/current profile is illustrated for a simulating, stimulating, amplifying, blocking and/or modulating electrical impulse applied to a portion or portions of the GI tract and/or GI nerves in accordance with an embodiment of the present invention.

Application of a suitable electrical voltage/current profile 400 for the simulating, stimulating, amplifying, blocking and/or modulating impulse 410 to the portion 200A of the GI tract and/or GI nerves, such as the vagus nerve 200, may be achieved using the electrical impulse generator 310. In a preferred embodiment, the electrical impulse generator 310 may be combined with a power source 320 and a control unit 330 having, for instance, a processor, a clock, a memory, etc., to produce a pulse train 420 to the electrodes 350 that deliver the simulating, stimulating, amplifying, blocking and/or modulating impulse 410 to the nerve 200 via leads 340.

The parameters of the modulation signal 400 are preferably programmable, such as the frequency, amplitude, duty cycle, pulse width, pulse shape, etc. In the case of an implanted ESDD device 300, programming of the control unit 330 may take place before or after implantation. For example, an implanted ESDD device 300 may have receiver 360 for communication of settings between the ESDD device 300 and programming unit 370. Programming unit 370 may include an external communication device to modify the programming of ESDD device 300 to improve treatment.

The impulse signal 410 preferably has a frequency, an amplitude, a duty cycle, a pulse width, a pulse shape, etc. selected to influence the therapeutic result, namely simulating, stimulating, amplifying, blocking and/or modulating some or all of the transmissions of sensations of satiety and hunger. The modulation signal may have a pulse width selected to influence the therapeutic result, such as about 20 μS or greater, such as about 20 μS to about 1000 μS. The modulation signal may have a peak voltage amplitude selected to influence the therapeutic result, such as about 1 mV or greater, such as about 1 mV to about 2 V.

In accordance with another embodiment, ESDD devices 300 in accordance with the present invention may be provided in a “pacemaker” type form, in which electrical impulses 410 are generated to a selected region 200A of the GI tract and/or GI tract nerves, such as the fundus region and/or vagus nerve 200, by ESDD device 300 on an intermittent basis to create in the patient a lower reactivity of the tissue or nerves to up-regulation signals, or to impart appropriate electrical impulses to dampen reactivity of the tissue or nerves to stimulus.

In all cases of permanent implantation, however, the implanting surgeon should vary the signal modulated by the control unit 330 and specific location of the electrode 350 until the desired outcome is achieved, and should monitor the long-term maintenance of this effect to ensure that adaptive mechanisms in the patient's body do not nullify the intended effects.

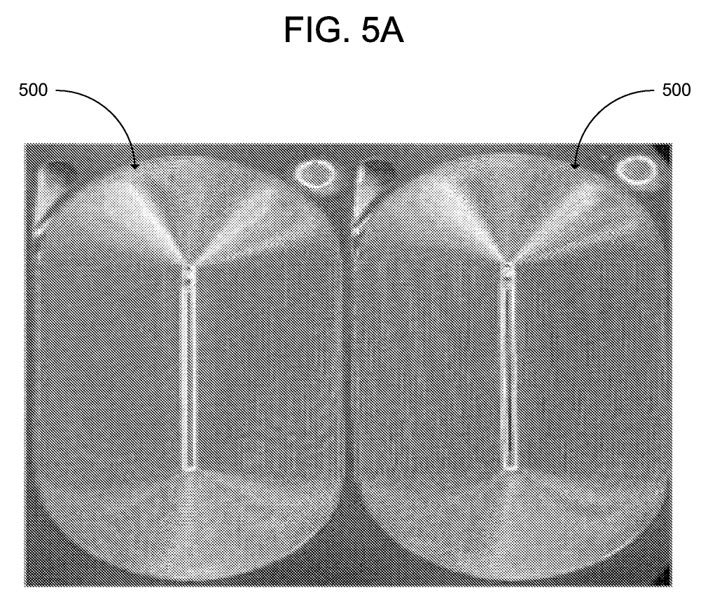

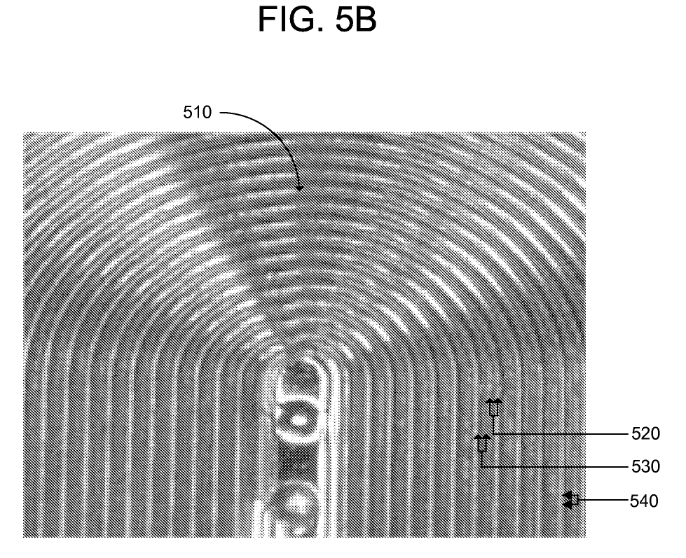

In accordance with a preferred embodiment of the present invention, the electrical stimulation treatment may be accomplished using sensing coils and treatment coils that capture and store the patient's natural signals (patient-generated signals, PGS). Micro-coils are commonly used for sensing applications. As discussed above, depending on the circumstances of treatment, one coil may be used for both sensing and modulating the patient's natural signals, while in other circumstances, a separate treatment coil or electrode may be preferable. Separate sensing and treatment coils may be preferable if the actions of sensing and modulating would be performed simultaneously. Coils preferably would be small for implantation, as shown in FIGS. 5A and 5B, and may be on a flexible substrate covered in implantable grade silicone or other material.

Referring to FIGS. 5A and 5B, an exemplary complex copper micro-coil 500 and a close-up thereof are illustrated for use in accordance with the present invention. As shown, each exemplary coil 500 has an overall width of 2.3 mm (0.090″) and length of 4.24 mm (0.167″). Each coil 500 has 44 turns 510. There are 4 coils 500 layered one over another and series wound for a total of 176 turns per induction system, such as an electrode 350. The illustrated conductor width 520 is 12.5 microns (0.0005″), and the illustrated spaces 530 between conductors are also 12.5 microns. The illustrated conductor height 540 is 7 microns (0.0003″). Each of the 4 copper conductor layers may be separated by a 10 micron (0.0004″) thick polyimide layer.

Exact details of wire size, turns and geometry of a sensing coil 500 of the present invention may be chosen to enable sensing of signals from 10-1000 Hz and 1 mV to 2 V. The microprocessor in the control unit 330 may use an analog to digital (A/D) converter to digitize the signal at a rate of 2000 samples/second or more and may store up to 500 seconds of it in memory (1 MB of memory). When required, this signal can be clocked out of the memory at the same rate and fed to a digital to analog (D/A) converter, amplified and applied to the patient through the treatment coil 500 and/or electrode 350. Additional background information may be found in U.S. Pat. No. 6,564,101 and U.S. Patent Application Number 20050222637, both of which are mentioned above and incorporated by reference (copies of which are attached hereto).

The sensing aspect of the present invention may utilize known sensing technology, such as that described in Familoni, U.S. Pat. No. 5,861,014, which is incorporated by reference. Familoni discloses an implantable pulse generator coupled to the gastric system and having a sensor, for sensing abnormalities in gastric electrical activity, and detecting means, for detecting abnormalities such as gastric arrhythmia, bradygastria, dysrhythmia, tachygastria, retrograde propagation, or uncoupling. If any of these gastric rhythm abnormalities is detected, then the pulse generator emits stimulation pulse trains to the gastric system to treat the detected gastric rhythm abnormalities.

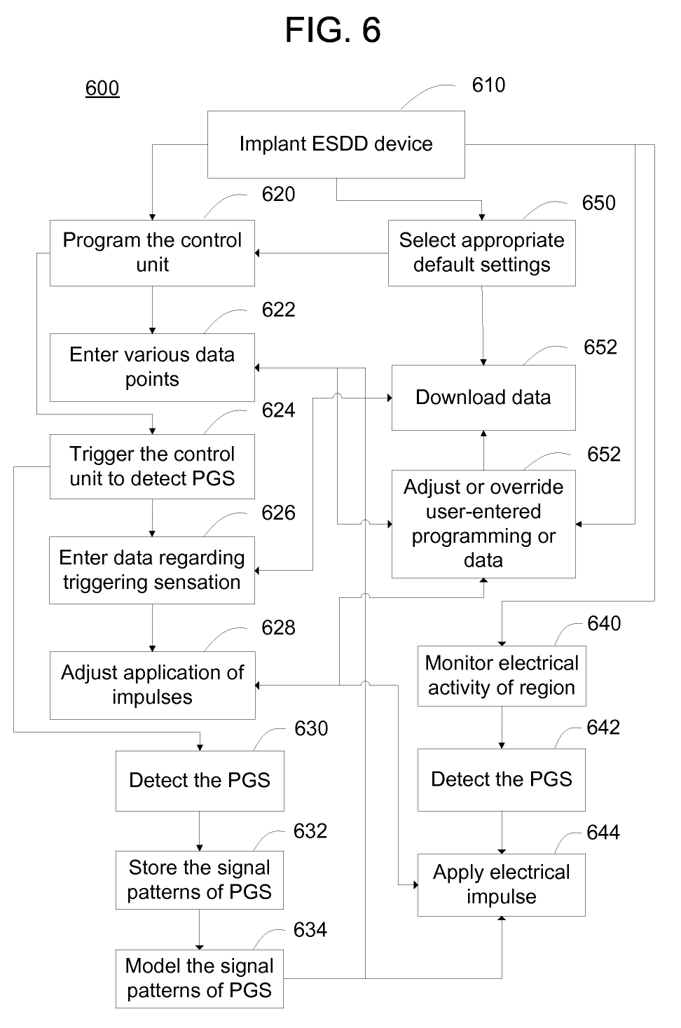

Referring to FIG. 6, a flow diagram of an exemplary implementation 600 of an embodiment of the present invention is illustrated. Connecting lines are for illustrative purposes only and shall not be used to limit the functionality of the present invention or imply a specific sequence of events. Many actions may occur in numerous orders and have no particular order.

In view of a patient's characteristics (gender, age, weight, height, health, etc.), an ESDD device 300 may be implanted (action 610) in the patient in the GI region where the best possible results are expected to be achieved. After implantation of the ESDD device 300 in a patient, the user 380 (the patient, a doctor, a healthcare attendant, etc.) may operate the programming unit 370 to program (action 620) the control unit 330.

Depending on the ESDD device configuration, the user 380 may enter (action 622) various data points as they occur, including mealtimes, meal durations, type and size of meal, meal contents, etc. In addition, when sensations affecting food consumption are felt by the patient, the user 370 (if not the patient, then in conjunction with the patient) may trigger (action 624) the programming unit 370 to detect or sense the sensation felt by the patient and may enter (action 626) the type of sensation and the perceived intensity of the sensation. The sensations may include sensations of hunger, sensations of satiety, sensations of stomach fullness, sensations of stomach emptiness, and sensations of stomach pain These data points comprise patient perceptions of various sensation-specific variables, such as sensation type, sensation time, sensation duration, and sensation strength. The control unit 330 may record the patient perceptions, such as for use in modeling the signals. The control unit 330 also may be pre-programmed to sense patient-generated signals (PGS) associated with such sensations, serving as an automatic trigger.

When triggered, the ESDD device 300 begins to detect (action 630) the PGS via the electrodes 350 and/or leads 340 and store (action 632) the signal patterns in the control unit 330. In conjunction with the data entered by the user 380 regarding the type and intensity of the sensation, the control unit 330 may associate the entered sensation type with the stored signal patterns of the PGS, as part of modeling (action 634) the PGS for a given sensation and intensity.

Based either on a pre-programmed model or a user-programmed model, the control unit 330 may monitor (action 640) the electrical activity of the GI tract tissue and/or GI tract nerves using the sensor means, to sense for various PGS associated with sensations affecting food consumption. When a PGS associated with a sensation affecting food consumption is detected (action 642) by the control unit 330, the control unit 330 may apply (action 644) an electrical impulse to simulate, stimulate, amplify, block and/or modulate the PGS. When appropriate, the control unit 330 takes no action.

For example, when a hunger PGS is detected in a patient needing to lose weight, control unit 330 may apply an electrical impulse to block or modulate down the hunger PGS, an electrical impulse to simulate a satiety PGS, or both. The intensity, duration and timing of the applied electrical impulses may be pre-programmed, subject to user-programming, or both. As examples, the user may be prompted as to whether the electrical impulse should be applied; a time delay may be incorporated into the programming; and times of day may be programmed during which the patient should eat, so time-appropriate hunger PGS would be unaffected.

The user may program (action 628) the control unit 330 in various ways, such as adjusting the application and intensity of hunger-related or satiety-related impulses. For instance, a patient may continue to feel hungry despite the circumstances, such as after eating a small meal, and the user may program the control unit 330 to apply an impulse simulating satiety PGS (which may be stimulating or amplifying an existing signal or signals) and/or blocking hunger PGS. Conversely, a patient feeling too full may program the control unit 330 to apply an electrical impulse blocking or modulating down the satiety PGS. Based on intervals between meals, the control unit 330 may apply an electrical impulse to amplify a detected PGS, either to maintain satiety in patients needing to lose weight, or to accelerate hunger in patients needing to gain weight.

Although device configuration limitations would bound the characteristics of the electrical impulses, in particular frequency and amplitude, that the control unit 330 would be able to apply, the device configuration limitations still may be beyond the ranges of impulses appropriate for patient treatment, so the ESDD device 300 may have therapeutic limitations pre-programmed into the control unit 330 that the user 380 could not override.

The ESDD 300 also may have pre-programmed default settings that an administrative user 390 may select (action 650), such as the physician, applicable to various patient characteristics and implantation arrangements. The administrative user 390 may exercise administrative rights, for example, via role-based access to the programming unit 370 or via an administrative unit, such as a personal computer to which the programming unit 370 may be connected.

Whenever necessary, an administrative user 390 furthermore may download (action 652) the data from the control unit 330 or the programming unit 370, depending on the ESDD device 300 configuration, to monitor patient progress and for review and revision of the treatment regime. As above, the download may occur on the programming unit 370 itself, allowing the administrative user 390 to review the data directly on the programming unit 370, such as if the programming unit 370 were a personal data assistant (“PDA”). Alternatively or in addition, the download may be to an administrative unit, such as for archival purposes. Likewise, as appropriate, the administrative user 390 may adjust or override (action 654) various programming and data entered by the user 380.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method, comprising:
quantifying one or more sensations identified by a patient relating to the patient's gastrointestinal (GI) tract based on information provided by the patient;
sensing activity of at least one of GI tissues and GI nerves of the patient;
correlating the sensed activity of the GI tissues and/or GI nerves with the patient-identified sensations relating to the patient's GI tract;
storing the patient-identified sensations, the sensed activity, and the correlation therebetween;
determining thereafter that one or more of the patient-identified sensations relating to the patient's GI tract are present based on further sensing activity of the GI tissues and/or the GI nerves of the patient; and
applying at least one electrical impulse to one or more selected regions of the GI tract of the patient to at least one of simulate, stimulate, amplify, block and modulate the activity of the GI tissues and/or the GI nerves of the patient to modify the sensations felt by the patient relating to the patient's GI tract.

2. The method of claim 1, wherein the modification of the sensations felt by the patient relating to the patient's GI tract are directed to the treatment of an eating disorder.

3. The method of claim 2, wherein the eating disorder includes at least one of overeating, overweight, obesity, and under-eating.

4. The method of claim 1, wherein the sensations identified by the patient are taken from the group consisting of: sensations of hunger, sensations of satiety, sensations of stomach fullness, sensations of stomach emptiness, and sensations of stomach pain.

5. The method of claim 1, wherein the sensing activity of the GI tissues and/or GI nerves of the patient are taken from the group consisting of: one or more muscles of the patient's GI tract, one or more nerves innervating the patient's GI tract, one or more nerves innervating the patient's fundus, one or more nerves innervating the patient's terminal branches of the left and right vagus nerves, and one or more nerves innervating one or more branches of the celiac plexus of the patient.

6. The method of claim 1, wherein the application of the at least one electrical impulse to the one or more selected regions of the GI tract of the patient is performed automatically upon the determination that the one or more patient-identified sensations are present.

7. The method of claim 6, wherein the automatic application of the at least one electrical impulse is subject to at least one of a time delay and a predetermined time interval.

8. The method of claim 6, wherein the automatic application of the at least one electrical impulse is subject to at least one of augmentation by and override by the patient.

9. A system, comprising:
input means for receiving information quantifying one or more sensations identified by a patient relating to the patient's gastrointestinal (GI) tract based on information provided by the patient;
sensing means for sensing activity of at least one of GI tissues and GI nerves of the patient;
processing means for correlating the sensed activity of the GI tissues and/or GI nerves with the patient-identified sensations relating to the patient's GI tract;
memory means for storing the patient-identified sensations, the sensed activity, and the correlation therebetween;
processing means for determining thereafter that one or more of the patient-identified sensations relating to the patient's GI tract are present based on further sensing activity of the GI tissues and/or the GI nerves of the patient; and
driving means for applying at least one electrical impulse to one or more selected regions of the GI tract of the patient to at least one of simulate, stimulate, amplify, block and modulate the activity of the GI tissues and/or the GI nerves of the patient to modify the sensations felt by the patient relating to the patient's GI tract.

10. An apparatus, comprising:
an electrical impulse generator;
a power source coupled to the electrical impulse generator;
a control unit in communication with the electrical impulse generator and coupled to the power source;
electrodes coupled to the electrical impulse generator; and
electrode leads or coils coupled to the electrodes for attachment to one or more selected regions of at least one of GI tissues and GI nerves of a patient;
wherein the control unit is operable to:
receive information quantifying one or more sensations identified by a patient relating to the patient's gastrointestinal (GI) tract based on information provided by the patient;
receive sensed activity of at least one of GI tissues and GI nerves of the patient from the electrode leads or coils;
correlate the sensed activity of the GI tissues and/or GI nerves with the patient-identified sensations relating to the patient's GI tract;
store the patient-identified sensations, the sensed activity, and the correlation therebetween;
determine thereafter that one or more of the patient-identified sensations relating to the patient's GI tract are present based on further sensing activity of the GI tissues and/or the GI nerves of the patient; and
cause the electrical impulse generator to apply at least one electrical impulse to one or more selected regions of the GI tract of the patient through the electrode leads or coils to at least one of simulate, stimulate, amplify, block and modulate the activity of the GI tissues and/or the GI nerves of the patient to modify the sensations felt by the patient relating to the patient's GI tract.
